SEQUENCE LISTING (5 sequences)

SEQ ID NO: 1 - DNA, 756 bp, Homo sapiens
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctccttttaacacggccctcaagctccttaagtga

SEQ ID NO: 2 - DNA, 21 bp, Artificial Sequence
  Forward primer for AIMP2
atgccgatgtaccaggtaaag

SEQ ID NO: 3 - DNA, 21 bp, Artificial Sequence
  Reverse primer for AIMP2
cttaaggagcttgagggccgt

SEQ ID NO: 4 - DNA, 24 bp, Artificial Sequence
  Forward primer for AIMP2-DX2
ctggccacgtgcaggattacgggg

SEQ ID NO: 5 - DNA, 20 bp, Artificial Sequence
  Reverse primer for AIMP2-DX2
aagtgaatcccagctgatag

(19) United States
(12) Patent Application Publication
(10) Pub. No.: US 2014/0142333 A1
(43) Pub. Date: May 22, 2014

(54) NOVEL ANILINE DERIVATIVES AND USE THEREOF

(71) Applicant: NEOMICS Co, Ltd., Suwon-si (KR)

(72) Inventors: Sunghoon KIM, Seoul (KR); Hee Sook Lee, Seoul (KR); Young Sun Oh, Seoul (KR); Dae Gyu Kim, Seoul (KR)

(73) Assignee: NEOMICS Co, Ltd., Suwon-si (KR)

(21) Appl. No.: 14/172,055

(22) Filed: Feb. 4, 2014

Related U.S. Application Data
(63) Continuation of application No. PCT/KR2012/006238, filed on Aug. 6, 2012.

(30) Foreign Application Priority Data
Aug. 4, 2011 (KR) 10-2011-0077863
Apr. 20, 2012 (KR) 10-2012-0041622

(51) Int. Cl. C07C 235/74; C07C 233/04; C07C 233/09

(52) U.S. Cl. CPC C07C 235/74; C07C 233/09; C07C 233/04
USPC 560/43; 564/155; 562/433

(57) ABSTRACT

Aniline derivatives for anticancer treatment including a compound of the Formula 1, or a derivative thereof, as an active ingredient,

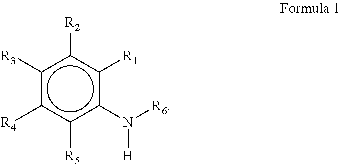

### CROSS-REFERENCE TO RELATED APPLICATIONS

This application is the continuation of International Application PCT/KR2012/006238, filed on Aug. 6, 2012, and claims priorities from and the benefit of Korean Patent Application No. 10-2011-0077863, filed on Aug. 4, 2011 and of Korean Patent Application No. 10-2012-0041622, filed on Apr. 20, 2012, all of which are incorporated herein by reference in their entireties for all purposes as if fully set forth herein.

### BACKGROUND

1. Field

The present invention relates to novel aniline derivatives or pharmaceutically acceptable salts thereof, and a pharmaceutical composition for preventing or treating cancer comprising the same.

2. Discussion of the Background

Through molecular and cellular analysis, it is known that genetic disruption of AIMP2 (ARS-interacting multi-functional protein 2) induces over-expression of c-myc, thereby hyper-proliferating alveolar epithelial cells of lung leading to neonatal lethality, and the expression of AIMP2 is induced by TGF-β, and inhibits expression of c-myc by being translocated into a nucleus (M. J. Kim, et. al., Nat. Genet. 34, 330-336, 2003).

Korean Patent Application No. 2005-110946 discloses that AIMP2 is a novel tumor suppressor, and has a function of enhancing signaling of TGF-β through direct interaction with Smad2/3, and in cancer cell lines and tissues, AIMP2-DX2, that is, exon 2-deleted splicing variant of AIMP2, is specifically expressed. Also, it was confirmed that in cells transformed with AIMP2-DX2, AIMP2 levels were dramatically reduced regardless of TGF-β, demonstrating that the generation of AIMP2-DX2 leads to a loss of AIMP2 activity. AIMP2-DX2 is closely associated with cancer formation and progression by inducing the decrease of AIMP2 levels. Accordingly, it was found that it is possible to diagnose various cancers such as lung cancer, liver cancer, skin cancer, breast cancer, renal cell carcinoma, and osteosarcoma, through generation of AIMP2-DX2. The patent application in its entirety is hereby cited by reference.

The AIMP2-DX2 protein is a splicing variant of AIMP2, in which in an AIMP2 protein sequence, an exon 2 region is deleted. The sequence of the AIMP2 protein (312aa version: AAC50391.1 or GI:1215669; 320aa version: AAH13630.1, GI:15489023, BC013630.1) is found in publications (312aa version: Nicolaides, N.C., et. al., Genomics 29 (2), 329-334 (1995)/320 aa version: Generation and initial analysis of more than 15,000 full-length human and mouse cDNA sequences, Proc. Natl. Acad. Sci. U.S.A. 99 (26), 16899-16903 (2002)). Korean Patent Application 10-2003-0018424, applied by the present inventors, discloses a cancer treatment effect of AIMP2 protein. The description on AIMP2 protein, in this patent publication, is hereby cited.

Also, when DNA is damaged, AIMP2 facilitates apoptosis by activating p53 (Han J M, et. al., Proc Natl Acad Sci USA, 105: 11206-11211 (2008)). It was examined that AIMP2-DX2 and AIMP2 competitively act while AIMP2-DX2 inhibits a pro-apoptosis function of AIMP2 through interruption of binding between AIMP2 and p53, causing cancer (Choi J W, et al., PLOS GENETICS, 7(3):e1001351, 2011). Thus, the publication describes that AIMP2-DX2 can be a novel antitumor agent target.

### SUMMARY

Accordingly, the present inventors have developed an antitumor agent capable of specifically controlling cancer without cytotoxicity, wherein the antitumor agent inhibits the expression of AIMP2-DX2 by degrading mRNA of AIMP2-DX2, and thus inhibits the growth of cancer cells. They found that the compound defined by Formula 1 in this specification shows the above described effect and thus is useful as an antitumor agent. Based on this finding, they completed this invention.

Accordingly, an object of the present invention is to provide an aniline derivative represented by Formula 1 or pharmaceutically acceptable salt thereof

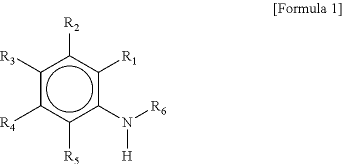

In the Formula 1:
R1 to R5 are each independently selected from the group consisting of a hydrogen, a straight, a branched, or cyclo alkyl of C1-C4, a halogen, an alkoxy, and a hydroxy;
R6 is

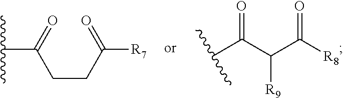

R7 is a hydroxy or

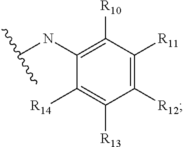

R8 is an alkoxy of C1-C6 or

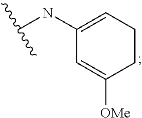 [SMILES: *NC1=CCCC(OC)=C1]

R9 is a hydrogen or an alkyl of C1-C6; and
R10 to R14 each independently selected from the group consisting of a hydrogen, a methyl, a halogen and a methoxy.

Another object of the present invention is to provide a pharmaceutical composition for preventing or treating cancer comprising the aniline derivative represented by Formula 1 or pharmaceutically acceptable salts thereof.

To achieve the objects, the present invention provides an aniline derivative represented by Formula 1 or pharmaceutically acceptable salt thereof.

To achieve other objects, the present invention provides a pharmaceutical compound for preventing or treating cancer comprising the aniline derivative represented by Formula 1 or pharmaceutically acceptable salts thereof.

The compounds Formula 1 may be selected from the below compounds:
4-[(3-ethoxy-1,3-dioxoprophyl)amino]benzoic acid;
N¹,N⁴-bis(3,4-dimethylphenyl)fumaramide;
N¹,N⁴-di-m-tolylfumaramide;
N¹-(2,5-dimethylphenyl)-N⁴-(3,4-dimethylphenyl)maleamide;
N¹,N⁴-di-m-tolylmaleamide;
N¹-(3,4-dimethylphenyl)-N⁴-(4-fluoro-2-methylphenyl)maleamide;
N¹-(3,4-dimethylphenyl)-N⁴-(3-fluoro-4-methylphenyl)maleamide;
N¹-(3,5-dichlorophenyl)-N⁴-(3,4-dimethylphenyl)maleamide;
(Z)-4-[(2,5-dimethylphenyl)amino]-4-oxobut-2-enoic acid;
(Z)-4-[(3,5-dimethylphenyl)amino]-4-oxobut-2-enoic acid;
(Z)-4-[(4-butylphenyl)amino]-4-oxobut-2-enoic acid;
(Z)-4-oxo-4-(m-tolylamino)but-2-enoic acid;
(Z)-4-[(4-fluorophenyl)amino]-4-oxobut-2-enoic acid;
(Z)-4-[(3,5-dichlorophenyl)amino]-4-oxobut-2-enoic acid;
(Z)-4-[(2,4-dichloro-6-methylphenyl)amino]-4-oxobut-2-enoic acid;
N¹-(3,4-dimethylphenyl)-N⁴-(3,5-dimethylphenyl)maleamide;
N¹-(3-butylphenyl)-N⁴-(3,4-dimethylphenyl)maleamide;
N¹-(4-bromophenyl)-N⁴-(3,4-dimethylphenyl)maleamide;
N¹-(4-fluorophenyl)-N⁴-(3-methoxyphenyl)maleamide;
N¹-(3-ethylphenyl)-N⁴-(4-fluorophenyl)maleamide;
(Z)-4-[(3-fluoro-4-methylphenyl)amino]-4-oxobut-2-enoic acid;
N¹,N⁴-bis(3,5-dichlorophenyl)fumaramide;
N¹,N⁴-bis(4-bromophenyl)fumaramide;
N¹,N⁴-bis(3,4-dichlorophenyl)fumaramide;
N¹,N⁴-bis(3-fluoro-4-methylphenyl)fumaramide;
N¹,N⁴-bis(4-methoxyphenyl)maleamide;
N¹-(3-fluoro-4-methylphenyl)-N⁴-(4-fluorophenyl)maleamide;
N¹,N⁴-bis(4-fluoro-2-methylphenyl)maleamide;
N¹-(2,5-dimethylphenyl)-N³-(3-methoxyphenyl)-2-methylmalonamide;
N¹-(4-fluorophenyl)-N⁴-(m-tolyl)maleamide;
N¹-(3,5-dimethylphenyl)-N³-(3-methoxyphenyl)-2-methylmalonamide;
ethyl 3-(anthracen-2-ylamino)-2-methyl-3-oxopropanoate;
ethyl 3-[(2-chloro-4-hydroxyphenyl)amino]-3-oxopropanoate; and
ethyl 3-[(2-chloro-4-hydroxyphenyl)amino]-2-methyl-3-oxopropanoate.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a location map of primer used the Example in the present invention.

FIG. 2 shows a schematic map of pGL2-DX2 vector for luciferase assay.

FIG. 3 shows western blot results indicating that the inventive compound specifically inhibits the expression of AIMP2-DX2 protein depending on concentration (BC-DXI01: 4-[(3-ethoxy-1,3-dioxopropyl)amino]-benzoic acid, Non: non-treating group). Tubulin is used as a positive control.

FIG. 4A shows western blot results indicating that the inventive compound specifically inhibits the expression of AIMP2-DX2 protein depending on time (BC-DXI01: 4-[(3-ethoxy-1,3-dioxopropyl)amino]-benzoic acid, Non: non-treating group). Tubulin is used as a positive control.

FIG. 4B shows RT PCR results indicating that the inventive compound specifically induces a degradation of AIMP2-DX2 mRNA transcript depending on time (BC-DXI01: 4-[(3-ethoxy-1,3-dioxopropyl)amino]-benzoic acid, Non: non-treating group). Actin is used as a positive control.

FIG. 5 shows RT PCR results indicating that the inventive compound specifically induces a degradation of AIMP2-DX2 mRNA transcript depending on time (BC-DXI01: 4-[(3-ethoxy-1,3-dioxopropyl)amino]-benzoic acid, Non: non-treating group). Actin is used as a positive control.

FIG. 6 shows test results of MTT assay indicating the inhibitory activity of the inventive compound on lung cancer cells.

FIG. 7 shows test results of FACS analysis indicating the effect of inducing apoptosis of the inventive compound on lung cancer cells.

FIG. 8 shows a test result that it was examined if the salt form of the inventive compound shows the same effect as the inventive compound (BC-DXI01: 4-[(3-ethoxy-1,3-dioxopropyl)amino]-benzoic acid, salt: salt form of BC-DXI01, On: BC-DXI01).

FIG. 9 shows a measurement result of a tumor volume of a mouse on which the inhibitory activity of the inventive compound on lung cancer was examined in vivo (G1: a control group not administered with 4-[(3-ethoxy-1,3-dioxopropyl)amino]-benzoic acid, G2: a group treated with 4-[(3-ethoxy-1,3-dioxopropyl)amino]-benzoic acid 50 mg/kg).

FIG. 10 shows a measurement result of a body weight of a mouse to confirm cytotoxicity of the inventive compound in vivo (G1: a control group not administered with 4-[(3-ethoxy-1,3-dioxopropyl)amino]-benzoic acid, G2: a group treated with 4-[(3-ethoxy-1,3-dioxopropyl)amino]-benzoic acid 50 mg/kg).

FIG. 11 shows a measurement result of a tumor weight of a mouse on which the inhibitory activity of the inventive compound on lung cancer was examined in vivo (G1: a control group not administered with 4-[(3-ethoxy-1,3-dioxopropyl)amino]-benzoic acid, G2: a group treated with 4-[(3-ethoxy-1,3-dioxopropyl)amino]-benzoic acid 50 mg/kg).

FIG. 12 shows a photograph of a result of an animal experiment after the inhibitory activity of the inventive compound on lung cancer cells were examined in vivo.

### DETAILED DESCRIPTION OF THE ILLUSTRATED EMBODIMENTS

Hereinafter, the present invention will be described in detail by examples. It is to be understood, however, that these examples are for illustrative purpose only and are not constructed to limit the scope of the present invention.

The term used in the present invention, “alkyl”, refers to a straight or branched saturated hydrocarbon radical, as long as it is not particularly defined.

The term used in the present invention, “halogen” or “halo”, refers to halogen atoms, and includes fluorine, chlorine, bromine, iodine, and the like.

The term used in the present invention, “alkoxy”, refers to O-alkoxy (alkyl is described above) as long as it is not particularly defined.

The term used in the present invention, “cycloalkyl”, refers to saturated hydrocarbon ring as long as it is not particularly defined.

Accordingly, an object of the present invention is to provide an aniline derivative represented by Formula 1 or pharmaceutically acceptable salt thereof.

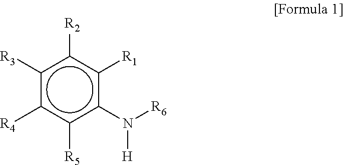

In Formula 1:

R1 to R5 are each independently selected from the group consisting of a hydrogen, a straight, a branched, or cyclo alkyl of C1-C4, a halogen, an alkoxy, and a hydroxy;

R6 is

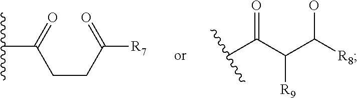

R7 is a hydroxy or

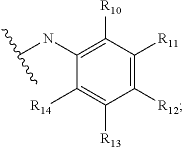

R8 is an alkoxy of C1-C6 or

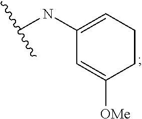 [SMILES: *NC1=CCCC(OC)=C1]

R9 is a hydrogen or an alkyl of C1-C6; and

R10 to R14 are each independently selected from the group consisting of a hydrogen, a methyl, a halogen and a methoxy.

The compound represented by Formula 1 of the present invention comprises a pharmaceutically acceptable salt. The pharmaceutically acceptable salt may be an addition salt formed from a inorganic acid or organic acid. Specifically, the salt may be an acid addition salt formed from a pharmaceutically acceptable free acid. The free acid may be an organic or inorganic acid. For the inorganic acid, hydrochloric acid, bromic acid, sulfuric acid and phosphoric acid can be used. For the organic acid citric acid, acetic acid, lactic acid, tartaric acid, fumaric acid, formic acid, propionic acid, oxalic acid, trifluoroacetic acid, methanesulfonic acid, benzensulfonic acid, maleic acid, benzoic acid, gluconic acid, glycolic acid, succinic acid, 4-morpholinethansulfonic acid, cam-phorsulfonic acid, 4-nitrobenzenesulfonic acid, hydroxy-O-sulfonic acid, 4-toluenesulfonic acid, caloktronic acid, amber acid, glutamic acid and aspartic acid.

The compound of Formula 1 in the present invention specifically could induce a selective degradation of AIMP2-DX2 mRNA transcript, thereby inhibiting the growth of cancer cells. While conventional antitumor agents mainly induce apoptosis by causing cytotoxicity, the compound can induce a degradation of oncogenic AIMP2-DX2 mRNA like siRNA. Thus, it was confirmed that the compound is useful as an antitumor agent for a novel mechanism, unlike a conventional antitumor agent.

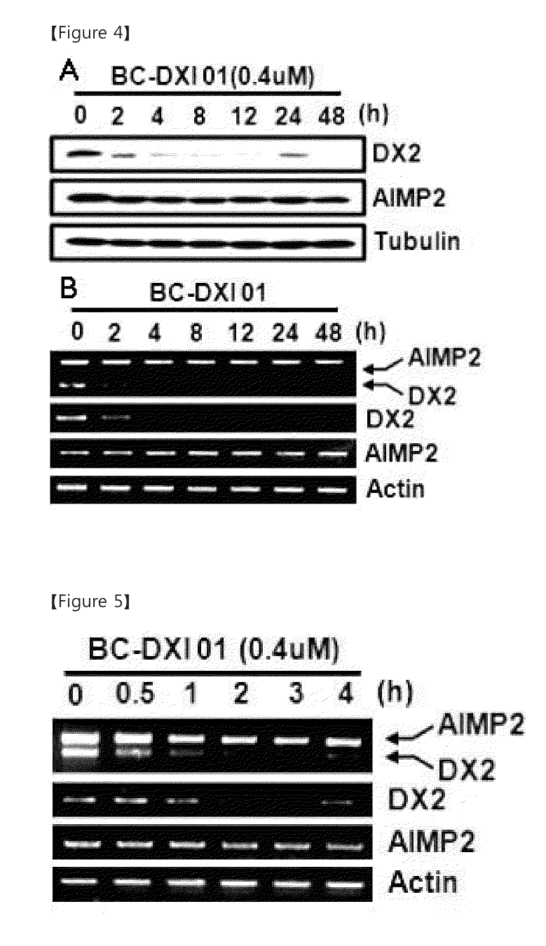

In one Example of the present invention, a compound inhibiting the growth of lung cancer cell was searched by treating the lung cancer cell line with various compounds. As a result, it was confirmed that one of the inventive compounds, 4-[(3-ethoxy-1,3-dioxopropyl)amino]-benzoic acid, reduces the level of AIMP2-DX2 and mRNA transcript of AIMP2-DX2 depending on treating time and concentration (FIG. 3, FIG. 4 and FIG. 5).

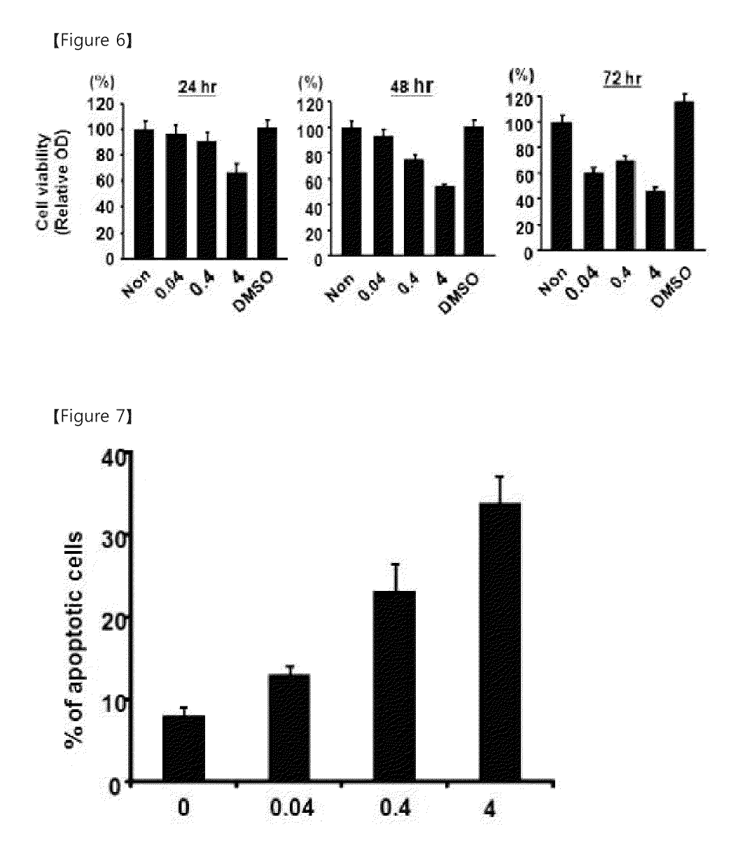

In another Example of the present invention, a lung cancer cell line was treated with 4-[(3-ethoxy-1,3-dioxopropyl)amino]-benzoic acid, and it was measured if the compound induces the death of lung cancer cells through MTT assay. As a result, it was confirmed that the lung cancer cells are subject to death, depending on treating time and concentration (FIG. 6). And the present inventor also confirmed that the compound could induce the apoptosis of lung cancer cell depending on concentration by FACS analysis (FIG. 7).

In another Example of the present invention, by using a mouse transplanted with a lung cancer cell line, it was examined if the inventive compound inhibits lung cancer, in vivo. As a result, it was confirmed that the inventive 4-[(3-ethoxy-1,3-dioxopropyl)amino]-benzoic acid effectively inhibits a tumor size of the mouse (FIG. 9 to FIG. 12).

In another Example of the present invention, various novel derivatives sharing an aniline structure with the inventive 4-[(3-ethoxy-1,3-dioxopropyl)amino]-benzoic acid were prepared (Table 1 to Table 7), and then it was examined if they show the same effect as 4-[(3-ethoxy-1,3-dioxopropyl)amino]-benzoic acid (Table 8 and Table 9).

As a result, it was confirmed that the inventive aniline derivatives effectively inhibit the activity of AIMP2-DX2 in cancer cells like 4-[(3-ethoxy-1,3-dioxopropyl)amino]-benzoic acid.

Accordingly, the inventors confirmed that aniline derivatives of the present invention effectively inhibit activity of cancer cells.

Therefore, the present invention provides a pharmaceutical composition for preventing or treating cancer comprising the aniline derivative represented by Formula 1 or pharmaceutically acceptable salts thereof.

The composition of the present invention preferably refers, but not limited thereto, a pharmaceutical composition. As used herein, “pharmaceutically acceptable” means a composition which is physiologically acceptable and, when administered to human beings, generally does not cause allergic reactions, such as gastrointestinal disorders and dizziness, or similar reactions thereto, as well as not inhibiting reaction of an active ingredient. A pharmaceutically acceptable carrier, for example, the carriers for the oral preparations may comprise lactose, starch, cellulose derivatives, magnesium stearate, stearic acid and the carriers for the parenteral preparations may comprise water, oil, saline, aqueous glucose and glycol and it may further comprise a stabilizer and a preservative.

The examples of the stabilizers may be sodium hydrogen sulfite, sodium sulfite, and ascorbic acid. The examples of the preservatives may be benzalkonium chloride, methyl- or prophyl-paraben, andchlorobutanol. The list of pharmaceutically acceptable carriers is disclosed in Remington's Pharmaceutical Sciences, 19th ed., Mack Publishing Company, Easton, Pa., 1995. The pharmaceutical composition of the present invention may be formulated into various reagents for oral administration or parenteral administration according to the method well known in the art. In case of parenteral administration, the composition may be formulated preferably into injections of isotonic solution or suspension. The injections may be prepared by the method well known in the art with a proper wetting agent or suspension agent. For example, each component may be dissolved into saline or buffer solution and formulated into injections. In addition, for oral administration, it may comprise, but not limited thereto, powders, granules, tablets, pills and capsules.

The pharmaceutical composition prepared by the above may be administered by various routes including oral, transdermal, intradermal, intravenous, and intramuscular administration. As used herein, “effective amount” refers to an amount of a composition or extract which exhibits the effect of preventing or treating a disease when it is administered into the patient. The dose of the pharmaceutical composition may be suitably determined by considering various factors, such as administering route, subject, age, sex, differences among individuals, and disease severity. Preferably, the anticancer composition may contain variable amount of effective ingredient according to the disease severity, but about 0.0001 μg to 10 kg of effective ingredient may be administered several times a day.

The anticancer composition of the present invention is very effective in treating cancer. The cancers comprise, but are not limited to, breast cancer, colon cancer, lung cancer, small cell lung cancer, stomach cancer, liver cancer, leukemia, bone cancer, pancreatic cancer, skin cancer, head or neck cancer, melanoma in skin or eyeball, uterine cancer, ovarian cancer, rectal cancer, anus cancer, oviduct cancer, endometrial carcinoma, cervical cancer, vagina cancer, Hodgkin's disease, esophagus cancer, small intestine cancer, endocrine gland cancer, thyroid cancer, parathyroid cancer, adrenal gland cancer, soft tissue sarcoma, urethra cancer, testis cancer, prostate cancer, chronic or acute leukemia, lymphocyte lymphoma, bladder cancer, kidney or ureter cancer, kidney cell carcinoma, kidney pelvis carcinoma, CNS tumor, primary CNS lymphoma, spinal cord tumor, brainstem glioma, pituitary adenoma or combinations thereof. In particular, it may be lung cancer.

Accordingly, a compound represented by Formula 1 of the present invention inhibits AIMP2-DX2, which is novel anticancer target and induces apoptosis of cancer cells, thereby being effective in preventing and treating cancer. Therefore, a compound of the present invention can be used for preventing and treating cancer.

### Example 1

### Screening for AIMP2-DX2 Inhibitor

In order to screen for a compound specifically inhibiting the activity of AIMP2-DX2, from a compound library bought from ChemDive (US), the inventors' transfected lung cancer cell line, H460, with pGL2-DX2 (see FIG. 2). After culturing the cell line for 24 hours, they treated it with a compound. Then, after further culturing for 4 hours, luciferase activity was measured through a luciferase assay kit according to a manufacturer protocol (Promega, US), by using a luminometer.

As a result, 22 compounds were primarily screened. Normal cells, that is, WI-26 cells were treated with the 22 compounds. After 48 hours, through MTT assay, a compound having no cytotoxicity was finally selected. As a result, the following compound of Formula 2 (4-[(3-ethoxy-1,3-dioxopropyl)amino]-benzoic acid) was selected (data not shown).

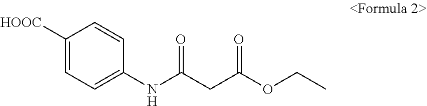 [SMILES: CCOC(=O)CC(=O)Nc1ccc(C(=O)O)cc1]

### Example 2

### Synthesis of 4-[(3-ethoxy-1,3-dioxopropyl)amino]-benzoic acid

Carboxylic acid of S1 (2.00 g, 14.9 mmol) and diethylmalonate of S2 (11.1 mL, 72.9 mmol) were mixed through stirring at 140° C. for 27 hours. The mixture was cooled to room temperature, and then left in boiling diethyl ether. The resultant mixture was cooled and filtered so as to obtain white powder of 4-[(3-ethoxy-1,3-dioxopropyl)amino]-benzoic acid (3.30 g, 92%).

Analysis results of NMR and MS are as follows.

1H NMR ((CD3)2SO, 300 MHz) δ 12.7 (s, 1H), 10.5 (s, 1H), 7.91 (d, J=8.7 Hz, 2H), 7.69 (d, J=8.6 Hz, 2H), 4.13 (q, J=7.1 Hz, 2H), 3.50 (s, 2H), 1.20 (t, J=7.1 Hz, 3H); MS(ES+) m/z calcd for C₁₂H₁₃NO₅(M+) 251.1. found 251.8

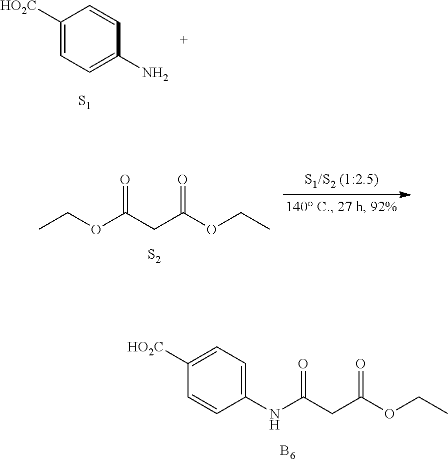

### Example 3

### Effect of the Inventive Compound on AIMP2-DX2 Activity

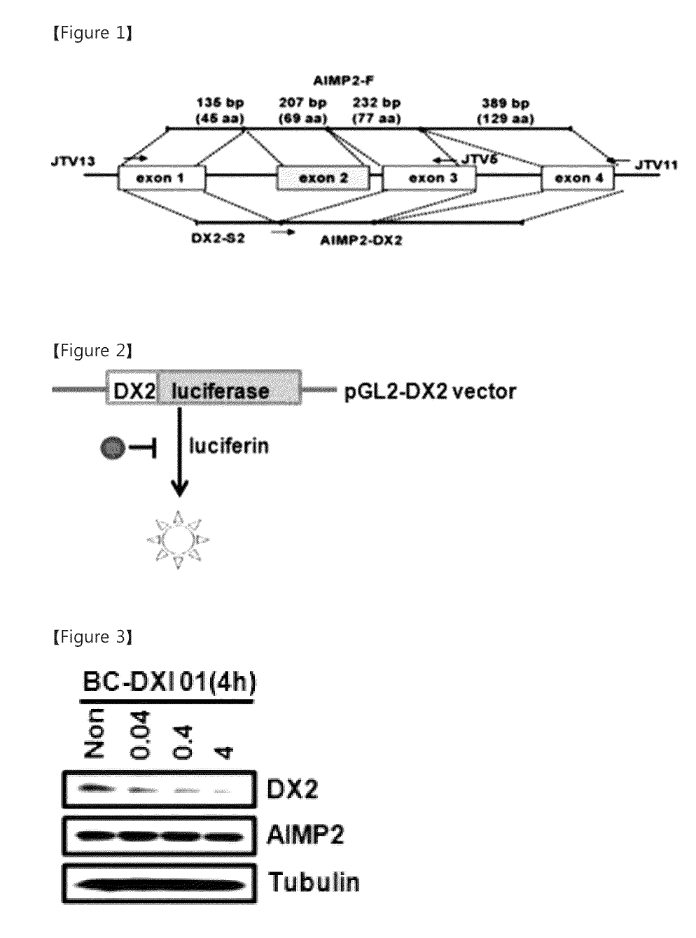

In order to investigate the effect of the compound of Formula 2 on activity of AIMP2-DX2, the present inventors performed western blotting and RT-PCR by using AIMP2 antibody and AIMP2-DX2 antibody (bought from Neomics (Korea)), and by using their specific primers shown FIG. 1.

RT-PCR was performed as follows.

Total RNAs were isolated following the protocol of the manufacturer (Qiagen). Freshly prepared tissues (3×3×3 mm) were chopped into small tissues, mixed with 350 ul lysis buffer, and homogenized using homogenizer or syringe. After adding 350 ul of 70% ethanol, the lysates were shaken upward and downward several times, loaded onto a column, and centrifuged at 13,000 RPM for 15 seconds. After washing the column with a wash buffer twice, RNAs were eluted with 40 ul of RNase-free DW. For reverse transcription, 1 μg of the isolated RNA was used as a template with the AIMP2-specific primer (SEQ ID NO:2 and SEQ ID NO:3) and DX2-specific primer (SEQ ID NO:4 and SEQ ID NO:5). After the reverse transcription, the mixture was diluted with DW 3 fold, and 1 ul of its aliquot was used for 30 ul PCR reaction containing 0.5 ul dMTP (2.5 mM each), 0.5 ul of primers indicated in FIG. 1 (JTV 13:SEQ ID NO:2, JTV 11:SEQ ID NO:3; DX2-S2:SEQ ID NO:4, JTV 5:SEQ ID NO:5) (each 10 pM), 1.5 ul of DMSO and 0.1 ul of Taq polymerase (5 U/μl).

Western blotting was performed as follows.

Cells were treated with the inventive compound for a predetermined time, and from the cells, proteins were extracted with protease-containing RIPA buffer, separated by 10 to 12% SDS-PAGE, and immuno-blotted with the specific antibodies using ECL system.

As a result, it was confirmed that expression of only AIMP2-DX2 protein was reduced dependently on the treatment time and concentration of the inventive compound, while the compound had no effect on expression of AIMP2 protein (see FIG. 3 and FIG. 4A).

Also, in order to examine the effect of the inventive compound on the degradation of AIMP2-DX2 mRNA depending on time, RT-PCR was performed. As a result, interestingly, it was confirmed that the inventive compound does not induce the degradation of AIMP2 mRNA, but specifically induces only the degradation of AIMP2-DX2 mRNA after 2 hr of treating the compound (See FIG. 4B).

In order to examine if the compound can degrade it in a shorter period of time, after the compound was treated for 30 minutes, 1 hour, 2 hours, 3 hours, and 4 hours, respectively, RT-PCR was performed. As a result, as shown in FIG. 5, it was confirmed that the inventive compound specifically degrades AIMP2-DX2 after 30 min of treating the compound. From the above described result, it can be seen that the inventive compound inhibits AIMP2-DX2 activity by degrading mRNA of AIMP2-DX2 as an antitumor agent target.

### Example 4

### In Vitro Inhibitory Effect of the Inventive Compound on Lung Cancer

<4-1> MTT Assay

The present inventors performed the following experiment in order to confirm the inhibitory effect of the inventive compound of Formula 2 on lung cancer.

Lung cancer cell line, NCI-H460, was cultured in RPMI (HyQ RPMI-1640, Hyclone) medium of streptomycin, containing 10% fetal bovine serum and 1% penicillin for 48 hours, and transferred to a 96-well plate. 12 hours later, the medium was replaced by serum free RPMI medium, and then the cell line was treated with the compound of Formula 1 at a concentration of 0.04 uM, 0.4 uM and 4 uM. 24 hours, 48 hours, and 72 hours later, MTT assay at each concentration was performed.

As a result, as shown in FIG. 6, it was confirmed that lung cancer cells were subject to cell death depending on treating time and concentration of the inventive compound.

<4-2> FACS Assay

Lung cancer cell line, NCI-H460, was cultured in RPMI (HyQ RPMI-1640, Hyclone) of streptomycin, containing 10% fetal bovine serum, and 1% penicillin. In order to investigate the effect of the inventive compound on a cell cycle, cells were treated with the inventive compound and cultured in medium containing 2% FBS. The cells were collected and subjected to FACS assay.

As a result, as shown in FIG. 7, it was confirmed that the treatment with the inventive compound concentration-dependently improves apoptosis rate of cancer cells.

<4-3> Test on the Effect of Salt Form of the Inventive Compound

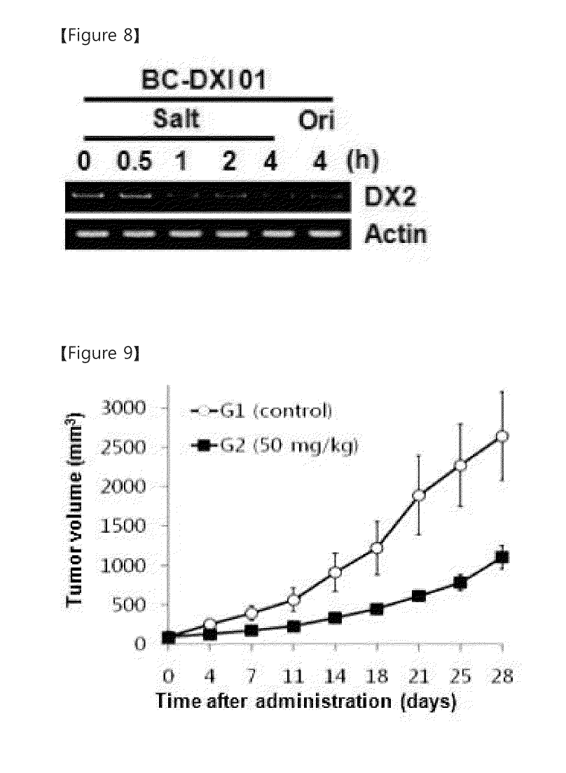

A salt form of the inventive 4-[(3-ethoxy-1,3-dioxopropyl)amino]-benzoic acid was prepared, and its inhibitory effect on AIMP2-DX2 was measured in the same manner as described in Example 3. As a result, as shown in FIG. 8, it was confirmed that the salt of the inventive compound induces the degradation of AIMP2-DX2 mRNA in lung cancer cells (H460 cells). Accordingly, it was found that the salt of the inventive compound effectively inhibits cancer cells.

### Example 5

### In Vivo Inhibitory Effect of the Inventive Compound on Lung Cancer

A nude mouse transplanted with NCI-H460 cells (human-derived lung cancer cell line) was administered with the salt form of 4-[(3-ethoxy-1,3-dioxopropyl)amino]-benzoic acid, through intra-abdominal cavity and intra-subcutaneous injection. Then, a growth inhibiting effect of a tumor was tested. Mice were divided into three groups such as a negative control group, and groups administered with a test material in doses of 50 and 100 mg/kg. Each group included 10 mice. The negative control group was administered with a mixture solution containing DMSO (excipient), Tween80, PEG400, and injection water, and the groups administered with the test material in doses of 50 mg/kg were administered with the inventive compound once a day, for 27 days including an autopsy day, 28 times in total (4 times for intra-abdominal cavity injection and 24 times for intra-subcutaneous injection.

During the observation period, general symptoms were observed once a day, and the body weight of an animal and the volume of a tumor were measured twice a week. On the day before autopsy, all individuals were fasted for 18 hours or more. On the day of autopsy, 0.5, 1 and 2 hours 1 after the test material was administered, from 3 mice, 3 mice and 4 mice from respective groups, blood was collected and tumor was extracted.

The collected blood was placed in an EDTA-containing tube, and centrifuged to separate plasma. The extracted tumor was weighed. Half of the plasma and the tumor were rapidly frozen by liquid nitrogen and placed in a frozen state, and the rest were fixed with 10% neutral buffered formalin solution and sent to a test client.

As a result, as shown in FIG. 9, it was confirmed that the volume of the tumor of the group treated with the inventive compound was significantly reduced, as compared to a control group.

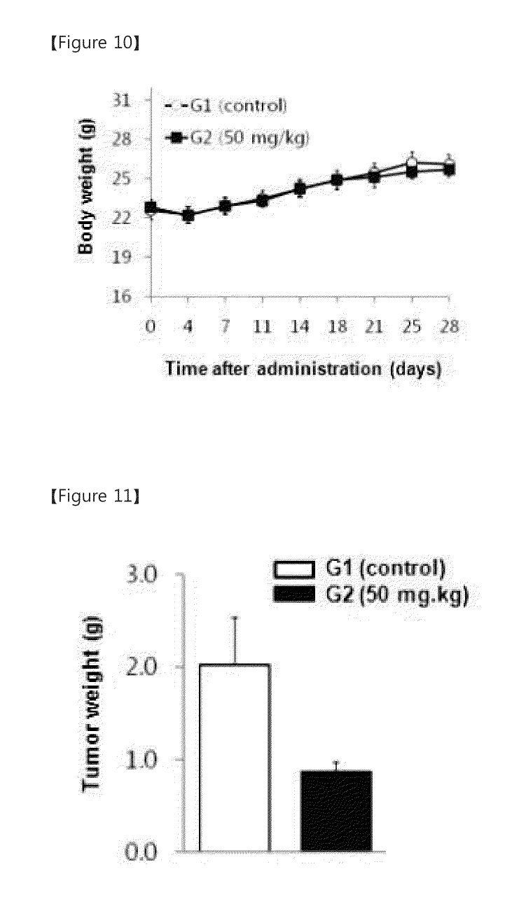

Also, as shown in FIG. 10, there is no difference in the body weight of a mouse between the control group and the group treated with the inventive compound. Thus, it was confirmed that the inventive compound has no toxicity.

Also, as shown in FIG. 11, it was confirmed that the weight of tumor in the group treated with the inventive compound was significantly reduced, as compared to that in the control group.

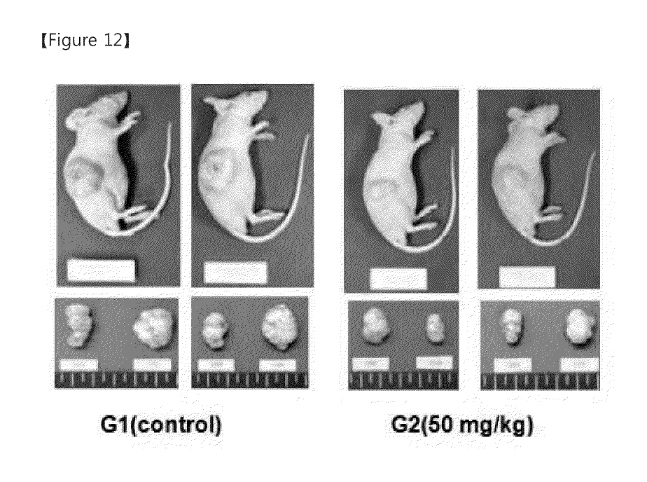

Also, tumors of mice in the group treated with the inventive compound and the control group were observed. As a result, as shown in FIG. 12, it was confirmed that the tumor in the group treated with the inventive compound was visibly significantly reduced.

### Example 6

### Inhibitory Effect of the Inventive Compound on Lung Cancer

Novel aniline derivatives having the similar structure as 4-[(3-ethoxy-1,3-dioxopropyl)amino]-benzoic acid showing the cancer inhibiting effect was synthesized (see Tables 1 to 7). In order to confirm their cancer inhibiting effect, in the same manner as described in Example 1, A549 and H460 (lung cancer cell line) were introduced with pGL-DX-2, cultured for 24 hours, and treated with the compound. After further culturing for 4 hours, luciferase activity was measured. On a negative control group (N.C) treated with DMSO instead of the inventive compound, and a positive control group (P.C) treated with 4-[(3-ethoxy-1,3-dioxopropyl)amino]-benzoic acid, the activities were measured.

As a result, as noted in Table 8 and Table 9, it was confirmed that the inventive novel aniline derivatives inhibit the level of AIMP2-DX2 unlike the control group, and thus are excellent in a cancer inhibiting effect like 4-[(3-ethoxy-1,3-dioxopropyl)amino]-benzoic acid.

TABLE 1

| No. | Structure | Name |
|---|---|---|
| 1 | 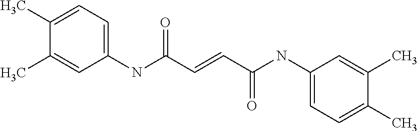 | N1,N4-bis(3,4- dimethylphenyl)fumaramide |
| 2 | 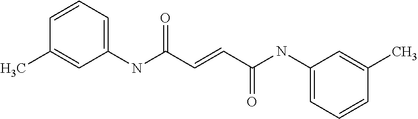 | N1,N4-di-m-tolylfumaramide |
| 3 | 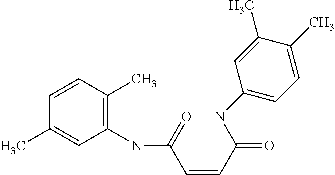 | N1-(2,5-dimethylphenyl)-N4- (3,4-dimethylphenyl) maleamide |
| 4 | 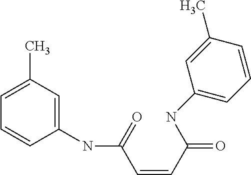 [SMILES: Cc1cccc(NC(=O)/C=C\C(=O)Nc2cccc(C)c2)c1] | N1,N4-di-m-tolylmaleamide |
| 5 | 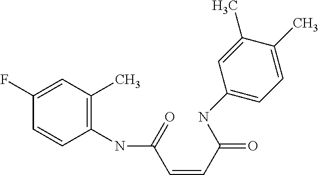 [SMILES: Cc1ccc(NC(=O)/C=C\C(=O)Nc2ccc(F)cc2C)cc1C] | N1-(3,4-dimethylphenyl)-N4- (4-fluoro-2-methylphenyl) maleamide |

TABLE 2

| | | |
|---|---|---|
| 6 | 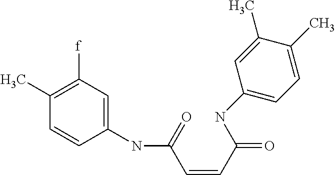 | N1-(3,4-dimethylphenyl)- N4-(3-fluoro-4- methylphenyl)maleamide |
| 7 | 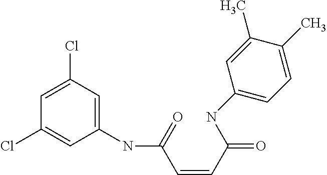 [SMILES: Cc1ccc(NC(=O)/C=C\C(=O)Nc2cc(Cl)cc(Cl)c2)cc1C] | N1-(3,5-dichlorophenyl)- N4-(3,4-dimethylphenyl) maleamide |
| 8 | 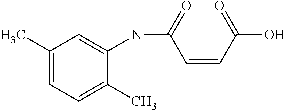 [SMILES: Cc1ccc(C)c(NC(=O)/C=C\C(=O)O)c1] | (Z)-4-[(2,5-dimethylphenyl) amino]-4-oxobut-2-enoic acid |
| 9 | 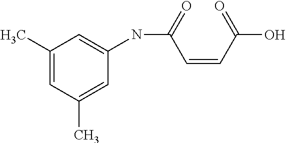 [SMILES: Cc1cc(C)cc(NC(=O)/C=C\C(=O)O)c1] | (Z)-4-[(3,5-dimethylphenyl) amino]-4-oxobut-2-enoic acid |
| 10 | 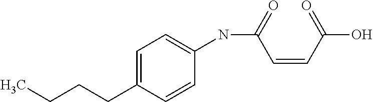 [SMILES: CCCCc1ccc(NC(=O)/C=C\C(=O)O)cc1] | (Z)-4-[(4-butylphenyl) amino]-4-oxobut-2- enoic acid |

TABLE 3

| No. | Structure | Name |
|---|---|---|
| 11 | 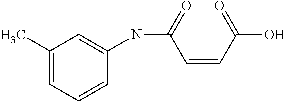 | (Z)-4-oxo-4-(m-tolylamino) but-2-enoic acid |
| 12 | 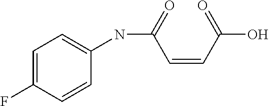 [SMILES: O=C(O)/C=C\C(=O)Nc1ccc(F)cc1] | (Z)-4-[(4-fluorophenyl) amino]-4-oxobut-2-enoic acid |
| 13 | 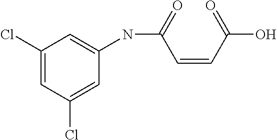 [SMILES: O=C(O)/C=C\C(=O)Nc1cc(Cl)cc(Cl)c1] | (Z)-4-[(3,5-dichlorophenyl) amino]-4-oxobut-2-enoic acid |
| 14 | 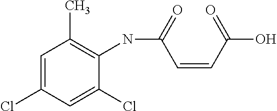 [SMILES: Cc1cc(Cl)cc(Cl)c1NC(=O)/C=C\C(=O)O] | (Z)-4-[(2,4-dichloro-6- methylphenyl)amino]-4- oxobut-2-enoic acid |
| 15 | 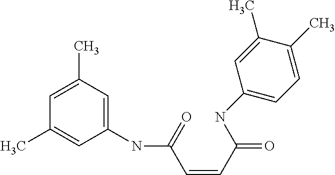 | N1-(3,4-dimethylphenyl)- N4-(3,5-dimethylphenyl) maleamide |

TABLE 4

| No. | Structure | Name |
|---|---|---|
| 16 | 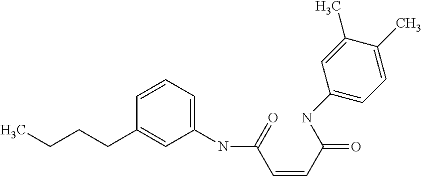 [SMILES: CCCCc1cccc(NC(=O)/C=C\C(=O)Nc2ccc(C)c(C)c2)c1] | N1-(3-butylphenyl)-N4-(3,4- dimethylphenyl)maleamide |
| 17 | 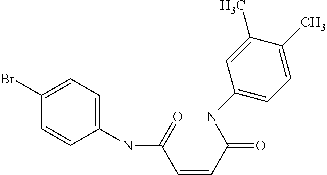 [SMILES: Cc1ccc(NC(=O)/C=C\C(=O)Nc2ccc(Br)cc2)cc1C] | N1-(4-bromophenyl)-N4-(3,4- dimethylphenyl)maleamide |
| 18 | 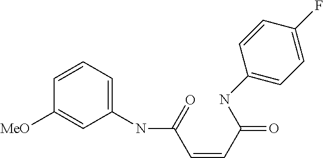 [SMILES: COc1cccc(NC(=O)/C=C\C(=O)Nc2ccc(F)cc2)c1] | N1-(4-fluorophenyl)-N4-(3,4- methoxyphenyl)maleamide |
| 19 | 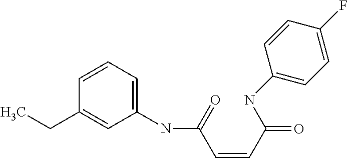 [SMILES: CCc1cccc(NC(=O)/C=C\C(=O)Nc2ccc(F)cc2)c1] | N1-(3-ethylphenyl)-N4-(4- fluorophenyl)maleamide |
| 20 | 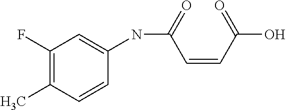 [SMILES: Cc1ccc(NC(=O)/C=C\C(=O)O)cc1F] | (Z)-4-[(3-fluoro-4- methylphenyl)amino]-4-oxobut- 2-enoic acid |

TABLE 5

| No. | Structure | Name |
|---|---|---|
| 21 | 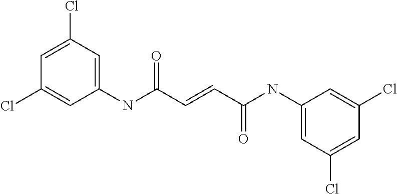 [SMILES: O=C(/C=C/C(=O)Nc1cc(Cl)cc(Cl)c1)Nc1cc(Cl)cc(Cl)c1] | N1,N4-bis(3,5- dichlorophenyl)fumaramide |
| 22 | 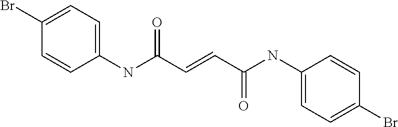 [SMILES: O=C(/C=C/C(=O)Nc1ccc(Br)cc1)Nc1ccc(Br)cc1] | N1,N4-bis(4 -bromophenyl) fumaramide |
| 23 | 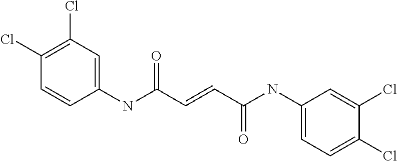 [SMILES: O=C(/C=C/C(=O)Nc1ccc(Cl)c(Cl)c1)Nc1ccc(Cl)c(Cl)c1] | N1,N4-bis(3,4- dichlorophenyl)fumaramide |
| 24 | 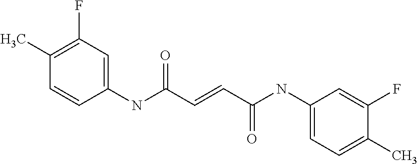 | N1,N4-bis(3-fluoro-4- methylphenyl)fumaramide |
| 25 | 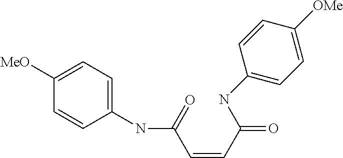 [SMILES: COc1ccc(NC(=O)/C=C\C(=O)Nc2ccc(OC)cc2)cc1] | N1,N4-bis(4-methoxyphenyl) maleamide |

TABLE 6

| No. | Structure | Name |
|---|---|---|
| 26 | 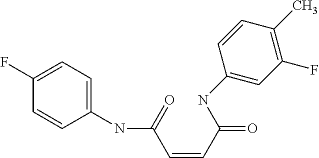 [SMILES: Cc1ccc(NC(=O)/C=C\C(=O)Nc2ccc(F)cc2)cc1F] | N1-(3-fluoro-4- methylphenyl)- N4-(4-fluorophenyl) maleamide |
| 27 | 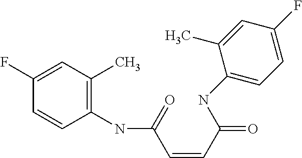 [SMILES: Cc1cc(F)ccc1NC(=O)/C=C\C(=O)Nc1ccc(F)cc1C] | N1,N4-bis(4-fluoro-2- methylphenyl)maleamide |
| 28 | 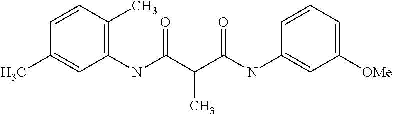 [SMILES: COc1cccc(NC(=O)C(C)C(=O)Nc2cc(C)ccc2C)c1] | N1-(2,5-dimethylphenyl)-N3- (3-methoxyphenyl)-2- methylmalonamide |
| 29 | 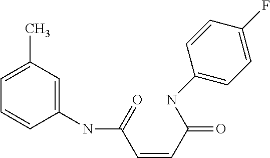 [SMILES: Cc1cccc(NC(=O)/C=C\C(=O)Nc2ccc(F)cc2)c1] | N1-(4-fluorophenyl)-N4-(m- tolyl)maleamide |
| 30 | 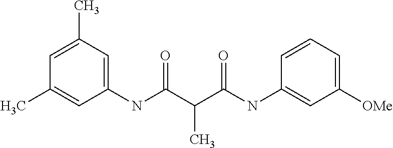 [SMILES: COc1cccc(NC(=O)C(C)C(=O)Nc2cc(C)cc(C)c2)c1] | N1-(3,5-dimethylphenyl)-N3- (3-methoxyphenyl)-2- methylmalonamide |

TABLE 7

| No. | Structure | Name |
|---|---|---|
| 31 | 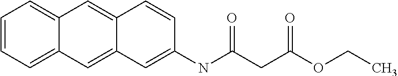 [SMILES: CCOC(=O)CC(=O)Nc1ccc2cc3ccccc3cc2c1] | ethyl 3-(anthracen-2-ylamino)-2- methyl-8-oxopropanoate |
| 32 | 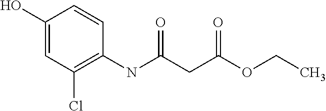 [SMILES: CCOC(=O)CC(=O)Nc1ccc(O)cc1Cl] | ethyl 3-[(2-chloro-4-hydroxyphenyl) amino]-3-oxopropanoate |
| 33 | 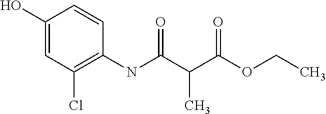 [SMILES: CCOC(=O)C(C)C(=O)Nc1ccc(O)cc1Cl] | ethyl 3-[(2-chloro-4-hydroxyphenyl) amino]-2-methyl-3- oxopropanoate |

TABLE 8

| | | AIMP2-DX2 | AIMP2-DX2 |
|---|---|---|---|
| | | activity in | activity in |
| No. | IUPAC Name | A549 cells | H460 cells |
| 1 | N1,N4-bis(3,4-dimethylphenyl)fumaramide | 14179 | 7101 |
| 2 | N1,N4-di-m-tolylfumaramide | 14072 | 7292 |
| 3 | N1-(2,5-dimethylphenyl)-N4-(3,4- | 10043 | 7749 |
| | dimethylphenyl)maleamide | | |
| 4 | N1,N4-di-m-tolylmaleamide | 9605 | 8189 |
| 5 | N1-(3,4-dimethylphenyl)-N4-(4-fluoro- | 9643 | 7427 |
| | 2-methylphenyl)maleamide | | |
| 6 | N1-(3,4-dimethylphenyl)-N4-(3-fluoro- | 10551 | 8901 |
| | 4-methylphenyl)maleamide | | |
| 7 | N1-(3,5-dichlorophenyl)-N4-(3,4- | 8268 | 5019 |
| | dimethylphenyl)maleamide | | |
| 8 | (Z)-4-[(2,5-dimethylphenyl)amino]- | 6705 | 7648 |
| | 4-oxobut-2-enoicacid | | |
| 9 | (Z)-4-[(3,5-dimethylphenyl)amino]- | 6786 | 7064 |
| | 4-oxobut-2-enoicacid | | |
| 10 | (Z)-4-[(4-butylphenyl)amino]-4- | 12399 | 8045 |
| | oxobut-2-enoicacid | | |
| 11 | (Z)-4-oxo-4-(m-tolylamino)but-2- | 6328 | 7143 |
| | enoicacid | | |
| 12 | (Z)-4-[(4-fluorophenyl)amino]-4- | 7937 | 7031 |
| | oxobut-2-enoicacid | | |
| 13 | (Z)-4-[(3,5-dichlorophenyl)amino]- | 8049 | 7855 |
| | 4-oxobut-2-enoicacid | | |
| 14 | (Z)-4-[(2,4-dichloro-6-methylphenyl)amino]- | 240 | 230 |
| | 4-oxobut-2-enoicacid | | |
| 15 | N1-(3,4-dimethylphenyl)-N4-(3,5- | 6981 | 8621 |
| | dimethylphenyl)maleamide | | |
| 16 | N1-(3-butylphenyl)-N4-(3,4-dimethylphenyl)maleamide | 6871 | 7511 |
| 17 | N1-(4-bromophenyl)-N4-(3,4-dimethylphenyl)maleamide | 6975 | 5718 |
| 18 | N1-(4-fluorophenyl)-N4-(3-methoxyphenyl)maleamide | 4239 | 3246 |

TABLE 9

| | | AIMP2-DX2 | AIMP2-DX2 |
|---|---|---|---|
| | | activity in | activity in |
| No. | IUPAC Name | A549 cells | H460 cells |
| 19 | N1-(3-ethylphenyl)-N4-(4-fluorophenyl)maleamide | 3978 | 3715 |
| 20 | (Z)-4-[(3-fluoro-4-methylphenyl)amino]- | 6446 | 7639 |
| | 4-oxobut-2-enoicacid | | |
| 21 | N1,N4-bis(3,5-dichlorophenyl)fumaramide | 7285 | 6061 |
| 22 | N1,N4-bis(4-bromophenyl)fumaramide | 5647 | 6377 |
| 23 | N1,N4-bis(3,4-dichlorophenyl)fumaramide | 5352 | 6391 |
| 24 | N1,N4-bis(3-fluoro-4-methylphenyl)fumaramide | 5812 | 7094 |
| 25 | N1,N4-bis(4-methoxyphenyl)maleamide | 5152 | 6085 |
| 26 | N1-(3-fluoro-4-methylphenyl)-N4- | 6525 | 6428 |
| | (4-fluorophenyl)maleamide | | |
| 27 | N1,N4-bis(4-fluoro-2-methylphenyl)maleamide | 5586 | 6029 |
| 28 | N1-(2,5-dimethylphenyl)-N3-(3- | 6691 | 7722 |
| | methoxyphenyl)-2-methylmalonamide | | |
| 29 | N1-(4-fluorophenyl)-N4-(m-tolyl)maleamide | 5662 | 5916 |
| 30 | N1-(3,5-dimethylphenyl)-N3-(3- | 7291 | 7167 |
| | methoxyphenyl)-2-methylmalonamide | | |
| 31 | ethyl | 5771 | 6346 |
| | 3-(anthracen-2-ylamino)-2-methyl- | | |
| | 3-oxopropanoate | | |
| 32 | ethyl | 19889 | 4848 |
| | 3-[(2-chloro-4-hydroxyphenyl)amino]- | | |
| | 3-oxopropanoate | | |
| 33 | ethyl | 18631 | 5403 |
| | 3-[(2-chloro-4-hydroxyphenyl)amino]- | | |
| | 2-methyl-3-oxopropanoate | | |
| P.C | 4-[(3-ethoxy-1,3-dioxopropyl)amino]- | 11651 | 5012 |
| | benzoic acid (Eaxample 1) | | |
| N.C | Add DMSO | 20089 | 8627 |

What is claimed is:

1. An aniline derivative represented by Formula 1, or a pharmaceutically acceptable salt of the aniline derivative: 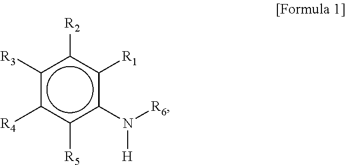
wherein:
R1 to R5 are each independently selected from the group consisting of a hydrogen, a straight, a branched or cyclo alkyl of C1-C4, a halogen, an alkoxy, and a hydroxy;
R6 is 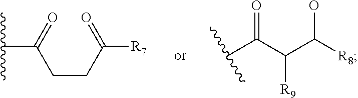
R7 is a hydroxy or 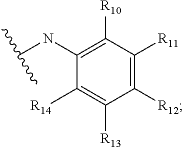
R8 is an alkoxy of C1-C6 or 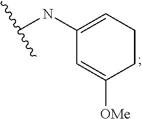 [SMILES: *NC1=CCCC(OC)=C1]
R9 is a hydrogen or an alkyl of C1-C6; and
R10 to R14 are each independently selected from the group consisting of a hydrogen, a methyl, a halogen, and a methoxy.

2. The aniline derivative of claim 1, wherein the aniline derivative is selected from the group consisting of:
N¹,N⁴-bis(3,4-dimethylphenyl)fumaramide;
N¹,N⁴-di-m-tolylfumaramide;
N¹-(2,5-dimethylphenyl)-N⁴-(3,4-dimethylphenyl)maleamide;
N¹,N⁴-di-m-tolylmaleamide;
N¹-(3,4-dimethylphenyl)-N⁴-(4-fluoro-2-methylphenyl)maleamide;
N¹-(3,4-dimethylphenyl)-N⁴-(3-fluoro-4-methylphenyl)maleamide;
N¹-(3,5-dichlorophenyl)-N⁴-(3,4-dimethylphenyl)maleamide;
*268(Z)-4-[(2,5-dimethylphenyl)amino]-4-oxobut-2-enoic acid;
(Z)-4-[(3,5-dimethylphenyl)amino]-4-oxobut-2-enoic acid;
(Z)-4-[(4-butylphenyl)amino]-4-oxobut-2-enoic acid;
(Z)-4-oxo-4-(m-tolylamino)but-2-enoic acid;
(Z)-4-[(4-fluorophenyl)amino]-4-oxobut-2-enoic acid;
(Z)-4-[(3,5-dichlorophenyl)amino]-4-oxobut-2-enoic acid;
(Z)-4-[(2,4-dichloro-6-methylphenyl)amino]-4-oxobut-2-enoic acid;
N¹-(3,4-dimethylphenyl)-N⁴-(3,5-dimethylphenyl)maleamide;
N¹-(3-butylphenyl)-N⁴-(3,4-dimethylphenyl)maleamide;
N¹-(4-bromophenyl)-N⁴-(3,4-dimethylphenyl)maleamide;
N¹-(4-fluorophenyl)-N⁴-(3-methoxyphenyl)maleamide;
N¹-(3-ethylphenyl)-N⁴-(4-fluorophenyl)maleamide;
(Z)-4-[(3-fluoro-4-methylphenyl)amino]-4-oxobut-2-enoic acid;
N¹,N⁴-bis(3,5-dichlorophenyl)fumaramide;
N¹,N⁴-bis(4-bromophenyl)fumaramide;
N¹,N⁴-bis(3,4-dichlorophenyl)fumaramide;
N¹,N⁴-bis(3-fluoro-4-methylphenyl)fumaramide;
N¹,N⁴-bis(4-methoxyphenyl)maleamide;
N¹-(3-fluoro-4-methylphenyl)-N⁴-(4-fluorophenyl)maleamide;
N¹,N⁴-bis(4-fluoro-2-methylphenyl)maleamide;
N¹-(2,5-dimethylphenyl)-N³-(3-methoxyphenyl)-2-methylmalonamide;
N¹-(4-fluorophenyl)-N⁴-(m-tolyl)maleamide;
N¹-(3,5-dimethylphenyl)-N³-(3-methoxyphenyl)-2-methylmalonamide;
ethyl 3-(anthracen-2-ylamino)-2-methyl-3-oxopropanoate;
ethyl 3-[(2-chloro-4-hydroxyphenyl)amino]-3-oxopropanoate; and
ethyl 3-[(2-chloro-4-hydroxyphenyl)amino]-2-methyl-3-oxopropanoate.

3. A pharmaceutical composition for preventing or treating cancer comprising an aniline derivative represented by Formula 1, or a pharmaceutically acceptable salt of the aniline derivative: 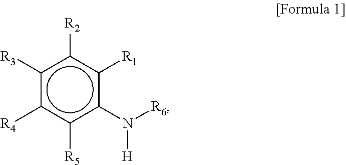
wherein:
R1 to R5 are each independently selected from the group consisting of a hydrogen, a straight, a branched or cyclo alkyl of C1-C4, a halogen, an alkoxy, a hydroxy, and a carboxyl;
R6 is 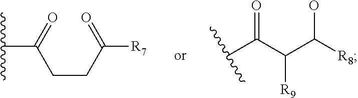
R7 is a hydroxy or 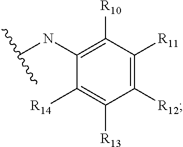
R8 is an alkoxy of C1-C6 or 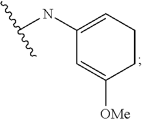 [SMILES: *NC1=CCCC(OC)=C1]
R9 is a hydrogen or an alkyl of C1-C6; and
R10 to R14 are each independently selected from the group consisting of a hydrogen, a methyl, a halogen, and a methoxy.

4. The pharmaceutical composition of claim 3, wherein the aniline derivative is selected from the group consisting of:
N¹,N⁴-bis(3,4-dimethylphenyl)fumaramide;
N¹,N⁴-di-m-tolylfumaramide;
N¹-(2,5-dimethylphenyl)-N⁴-(3,4-dimethylphenyl)maleamide;
N¹,N⁴-di-m-tolylmaleamide;
N¹-(3,4-dimethylphenyl)-N⁴-(4-fluoro-2-methylphenyl)maleamide;
N¹-(3,4-dimethylphenyl)-N⁴-(3-fluoro-4-methylphenyl)maleamide;
N¹-(3,5-dichlorophenyl)-N⁴-(3,4-dimethylphenyl)maleamide;
*268(Z)-4-[(2,5-dimethylphenyl)amino]-4-oxobut-2-enoic acid;
(Z)-4-[(3,5-dimethylphenyl)amino]-4-oxobut-2-enoic acid;
(Z)-4-[(4-butylphenyl)amino]-4-oxobut-2-enoic acid;
(Z)-4-oxo-4-(m-tolylamino)but-2-enoic acid;
(Z)-4-[(4-fluorophenyl)amino]-4-oxobut-2-enoic acid;
(Z)-4-[(3,5-dichlorophenyl)amino]-4-oxobut-2-enoic acid;
(Z)-4-[(2,4-dichloro-6-methylphenyl)amino]-4-oxobut-2-enoic acid;
N¹-(3,4-dimethylphenyl)-N⁴-(3,5-dimethylphenyl)maleamide;
N¹-(3-butylphenyl)-N⁴-(3,4-dimethylphenyl)maleamide;
N¹-(4-bromophenyl)-N⁴-(3,4-dimethylphenyl)maleamide;
N¹-(4-fluorophenyl)-N⁴-(3-methoxyphenyl)maleamide;
N¹-(3-ethylphenyl)-N⁴-(4-fluorophenyl)maleamide;
(Z)-4-[(3-fluoro-4-methylphenyl)amino]-4-oxobut-2-enoic acid;
N¹,N⁴-bis(3,5-dichlorophenyl)fumaramide;
N¹,N⁴-bis(4-bromophenyl)fumaramide;
N¹,N⁴-bis(3,4-dichlorophenyl)fumaramide;
N¹,N⁴-bis(3-fluoro-4-methylphenyl)fumaramide;
N¹,N⁴-bis(4-methoxyphenyl)maleamide;
N¹-(3-fluoro-4-methylphenyl)-N⁴-(4-fluorophenyl)maleamide;
N¹,N⁴-bis(4-fluoro-2-methylphenyl)maleamide;
N¹-(2,5-dimethylphenyl)-N³-(3-methoxyphenyl)-2-methylmalonamide;
N¹-(4-fluorophenyl)-N⁴-(m-tolyl)maleamide;
N¹-(3,5-dimethylphenyl)-N³-(3-methoxyphenyl)-2-methylmalonamide;
ethyl 3-(anthracen-2-ylamino)-2-methyl-3-oxopropanoate;
ethyl 3-[(2-chloro-4-hydroxyphenyl)amino]-3-oxopropanoate; and
ethyl 3-[(2-chloro-4-hydroxyphenyl)amino]-2-methyl-3-oxopropanoate.

5. The pharmaceutical composition of claim 3, wherein the cancer is selected from the group consisting of breast cancer, colon cancer, lung cancer, small cell lung cancer, stomach cancer, liver cancer, leukemia, bone cancer, pancreatic cancer, skin cancer, head or neck cancer, melanoma in skin or eyeball, uterine cancer, ovarian cancer, rectal cancer, anus cancer, oviduct cancer, endometrial carcinoma, cervical cancer, vagina cancer, Hodgkin's disease, esophagus cancer, small intestine cancer, endocrine gland cancer, thyroid cancer, parathyroid cancer, adrenal gland cancer, soft tissue sarcoma, urethra cancer, testis cancer, prostate cancer, chronic or acute leukemia, lymphocyte lymphoma, bladder cancer, kidney or ureter cancer, kidney cell carcinoma, kidney pelvis carcinoma, CNS tumor, primary CNS lymphoma, spinal cord tumor, brainstem glioma, and pituitary adenoma.

6. The pharmaceutical composition of claim 3, wherein the cancer is lung cancer.
